(19) United States
(12) Patent Application Publication
(10) Pub. No.: US 2020/0048179 A1
(43) Pub. Date: Feb. 13, 2020

(54) METHOD OF PRODUCING PLANT BIOMASS-BASED BIOPLASTIC

(71) Applicant: Kaien Yang, Chantilly, VA (US)

(72) Inventor: Kaien Yang, Chantilly, VA (US)

(21) Appl. No.: 16/101,435

(22) Filed: Aug. 11, 2018

(51) Int. Cl. C07C 67/08; C11C 3/00; B01J 23/04; C11B 1/10

(52) U.S. Cl. CPC C07C 67/08; C11B 1/10; B01J 23/04; C11C 3/003

(57) ABSTRACT

This invention relates to the field of producing bioplastics. Specifically, it relates to a method of producing all key ingredients of bioplastic making from pumpkins and making of bioplastic with these ingredients. More specifically, glycerin and other chemicals are extracted from pumpkin seed oil and mixed with starches that in the pumpkin flesh to make bioplastic. The bioplastic produced with the method as disclosed in this invention possess superior properties in tensile strength and biodegradability compared to bioplastic.

### TECHNOLOGY FIELD

A method of producing bioplastic from plant derived biomass such as pumpkins. The method includes separating pumpkin flesh and seeds, extracting organic glycerin and other chemicals from the seeds, and mixing with pumpkin flesh to form bioplastics.

### TECHNOLOGY BACKGROUND

Plastic and Plastic Waste

Plastic is a carbon-rich raw material consisting of any of a wide range of synthetic or semi-synthetic organic compounds that are malleable and so can be molded into any forms. Most of the plastics produced in the world is made from petrochemicals. In the process of producing plastics, crude oil is first refined into ethane, propane and other intermediates, from which ethylene and propylene are transformed under a high temperatures. In the process, plastic polymers will be formed from these monomers with the presence of catalysts. Because of the natural degradation of plastics takes a very long time, as a result, plastic waste is ubiquitous in every corner of the environment. Moreover, petroleum is a non-renewable commodity. The environmental concern long drove the need for producing biodegradable plastic from a renewable source.

Bioplastics and Biopolymers

In the current art, multiple methods exist to produce bioplastics from renewable biomass such as carbohydrate, protein or polyesters.

Carbohydrate Based Bioplastics

Starch and cellulose are common starting carbohydrates for bioplastics. Starch, when adding with additives, can be processed thermo-plastically. The addition of other biodegradable polyesters can improve its malleability. Starch based bioplastics account approximately half of the bioplastics in the market. Despite its abundance and versatility, significant challenge still exists to increase the physical properties of starch-based composites. Moreover, many starch based plastic shows less favored biodegradability. Cellulose, a structural component in plant cell wall, is a polysaccharide consisting of a linear chain of several hundred to many thousands of (C₆H₁₀O₅)ₙ units. Cellulose film or paper had a long history of application in various industries. The hydroxyl groups of cellulose can partially or fully react with various reagents to produce cellulose esters which can then be produced into bioplastic. The main disadvantage of cellulose based bioplastic its hydrophilic nature. Very often, the plastic made from cellulose possess low water vapor barrier and have poor process ability. It also has high brittleness, limited long-term stability and poor mechanical properties.

Polyester Polylactic Acid (PLA) Based Bioplastic

Another commonly used plant based raw material is PLA. PLA derived from lactic acid which is a fermented byproduct from dextrose which is in turn derived from many plants, mainly corn. It is a thermoplastic, biodegradable aliphatic polyester having potential for packaging applications. The lactic acid monomers are either directly poly-condensed or undergo ring-opening polymerization of lactide to form PLA pellets. PLA is the first bio-based polymer commercialized on a large scale and replaced high-density polyethylene, low-density polyethylene (LDPE), and polyethylene terephthalate (PETP) as packaging material in certain degree. However, it exhibits inferior impact strength, thermal robustness, and barrier properties.

### PROBLEM OF THE EXISTING METHOD

Despite multiple methods of producing bioplastic, significant hurdles exist in achieving the goal of replacing petroleum-based plastic. First, not all starting organic materials in current methods come from plant sources. The currently available bioplastic still carries heavy carbon footprint. Second, Existing methods produce bioplastics from maize, sugarcane, wheat and other crops. It had been reported that bioplastics had contributed to the global food crisis by taking over large areas of land previously used to grow crops for human consumption. In summary, there is an urgent need for a cost effective method to synthetize biodegradable bioplastic from entire plant source.

Pumpkins as a Source for Bioplastic

Pumpkin is constantly wasted each year, making it an inexpensive feedstock for bioplastics. On average, the U.S. produces 1.9 billion pounds of pumpkin each year, with approximately 80 percent ended up in the landfill, causing greenhouse gas emissions. A reverse logistic solution is possible to collect otherwise wasted pumpkins to a centralized location and offer a commercially viable alternative to petroleum-based plastics. In addition, pumpkins have a composition of carbohydrate and polyester that is favorable to bioplastic production.

### BRIEF DESCRIPTION OF THE DRAWINGS

FIG. 1 is a schematic representation showing a method of the producing bioplastic with two key components derived both from pumpkins.

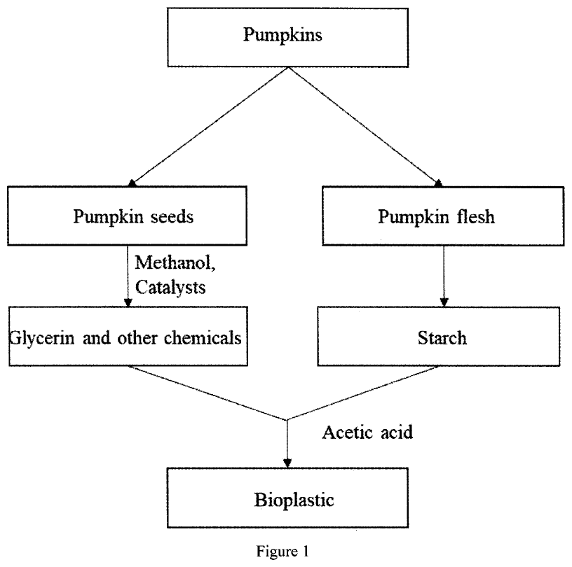

FIG. 2 shows tensile strength comparison result between bioplastics made with pumpkins seed derived glycerin and petroleum derived glycerin.

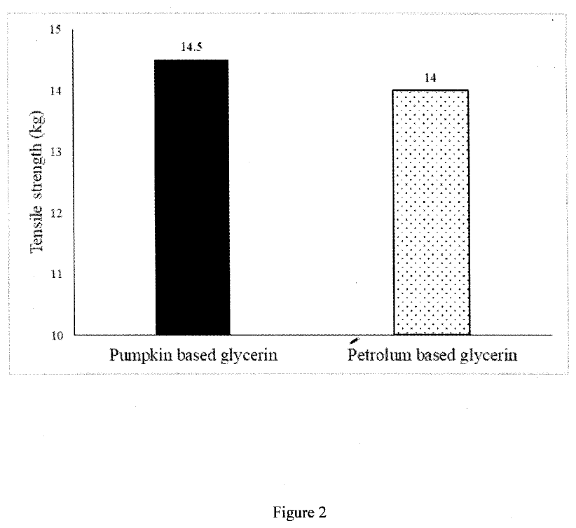

FIG. 3 shows biodegradability comparison result between bioplastics made with pumpkins seed derived glycerin and petroleum derived glycerin.

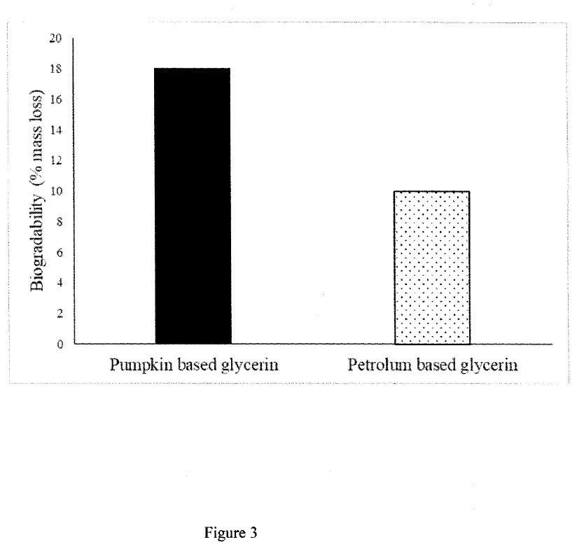

FIG. 4 summaries the comparison of four catalysts used in bioplastic production.

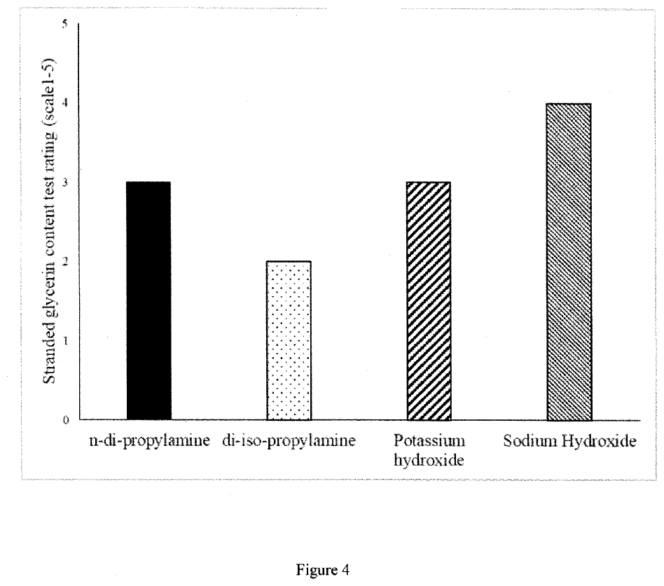

### DETAILED DESCRIPTION

The following disclosure covers the method of producing bioplastic from pumpkins. Specific details of the process are provided in examples for understanding of preferred embodiment.

Mechanical Separation of Pumpkins into Flesh and Seeds

To begin with, pumpkin seeds are separated from pumpkin flesh. The pumpkin flesh is ground until it is a near-homogenous mixture with minimal surface disruptions. An optional step may be applied to remove pumpkin rind from the flesh before grounding. Pumpkin seeds are separately cleaned and dried. The de-shelled seeds are subject to a mechanic extraction apparatus such as an oil extractor for seed oil. The pumpkin seeds are crushed inside the machine, and the seed oil is flowing out through the outlet the machine. The oil is collected after filtration.

The Transesterification Procedure of Making Organic Glycerin

Pumpkin seed oil will be heated to a range between 20° C. and 70° C., such as a temperature about 45° C.-55° C. Appropriate amount of methanol, between one fifth and two fifths of the pumpkin seed oil, by volume, will be measured and dispensed into a glass container. Appropriate catalyst may be added to methanol. In one embodiment, sodium hydroxide, between 1/100 to 1/200 (weight/volume) of methanol, will be added to methanol with gentle shaking until the catalyst completely dissolved in the methanol. The methanol/catalyst mixture will then be added to pumpkin seed oil and kept in an environment between 15° C. and 35° C. for fourteen days. Two definitive phases will be formed at the end of 14 days. The top layers if consisting of biodiesel, and the bottom layers includes glycerin and other chemicals.

Glycerin can be separated from biodiesel by a separating funnel into a collecting container. The bumpkin seed derived glycerin will be used in bioplastic synthesis in the next step.

Bioplastic Process

Bioplastics, like all plastics, contain at least one polymer and at least one plasticizer. Polymers contain extensive repeating groups of monomers and often feature a long carbon chain. A plasticizer is an additive that increases the pliability or flexibility of the plastic. In the pumpkin-based bioplastic, pumpkin flesh is used as the main polymer because it contains high level of starch, itself a repeating units of glucose and other monosaccharides. The primary composition of starch in pumpkin flesh is amylose which is a linear molecule that connects through α(1-4) bound to glucose molecules, and amylopectin, a branched molecule broken at α(1-6) position. When exposed to heat, the amylose and the amylopectin in starch became more organized (linear) due to hydrogen bonding between the hydroxyl groups. This linearization contributes to the stiffness of the bioplastic. The ratio of amylose to amylopectin somewhat determines the bioplastic's flexibility; a higher ratio (more amylose) results in a more rigid bioplastic, whereas a lower ratio (more amylopectin) results in a more flexible bioplastic. Most plant starches contain 20 percent amylose and 80 percent amylopectin.

Glycerin extracted from pumpkin seed oil will be used as a plasticizer because it contained polar hydroxyl groups at 1.51 Debye which absorbed water. Acetic acid is used to strengthen the bonds between the plasticizer and the polymer. The ions that are present in acetic acid dissociate the polymer, making it readily dissolvable. Other chemicals the present in the pumpkin seed oil that co-extracted with glycerin improved the performance bioplastic end product such as tensile strength and biodegradability. Other aspects and advantage of this invention are discussed in the embodiment with reference of drawing.

### Embodiment Examples

An embodiment of this invention is given with reference to description of the drawings.

1,000 grams of dry pumpkin seeds are peeled and placed in an oil extractor to extract oil. Extracted oil will be subject to a stainless steel screen with mesh size 100 micron. 200 ml of extracted pumpkin seed oil will be heated to 50° C. 0.4 grams of sodium hydroxide will be added to 44 mL of methanol and mixed gently until it is fully dissolved. The methanol is then added to the pumpkin seed oil in 50° C. with gentle shaking. The mixing bottle was left alone at 25° C. for fourteen days until two definitive layers of glycerin and biodiesel was observed. The top layers is consisting of biodiesel, and the bottom layers includes glycerin and other chemicals.

Glycerin can be separated from biodiesel by a separating funnel into a collecting container. The bumpkin seed derived glycerin will be used in bioplastic synthesis in the next step. Typically, 30-55 ml glycerin will be recovered.

100 grams of pumpkin flesh with rind removed are ground until it is a near-homogenous mixture with minimal surface disruptions. 65 mL of the pumpkin mixture was poured into a glass container. 10 mL of glycerin from the previous step and 10 mL of acetic acid (5% concentration) were also poured into pumpkin mixture and heated to 93° C. 120 mL of deionized water will be added while remained at 93° C. for 10 minutes. Afterwards, the contents of the will be poured into the tray to a designed thickness and left to cool at room temperature. In one example, 100 ml of mixture will be poured into a 12×12 inch tray and bioplastic film is formed in the process. For every 11 hours, the bioplastic can be lightly heated to expedite the drying process.

After harvested from the tray, the bioplastic film is undergone a series of testing for its strength and biodegradability to compare with plastic made with petroleum derived glycerin.

Bioplastic Strength Test and Result

The testing apparatus was made with the plastic samples sandwiched between two strips of wood and secured with two clamps. The wood was rested on two elevated platforms. An S-hook was latched onto the opening of the resting clamp, and to a chain underneath the clamp. The chain connected the handle of the bucket and the S-hook. The containers were used to gravel pour in the suspended bucket until the bioplastic ripped. The plastic's weight was recorded and the test was repeated for the different plastics. The result suggests that bioplastic, with tensile strength of 14.5 kg, outperformed petroleum-based glycerin, and tensile strength of 14 kg.

Bioplastic Biodegradability Test

In a plastic container that functioned as a composter, layers of garden soil and other compost materials were prepared to simulate plastic natural degradation environment for plastics. There are two layers of composts; a brown layer that is comprised of carbon-rich items including dry fall leaves, sticks, and bark, and a green layer was made of the green materials that were damp and high in nitrogen. For green layers, damp summer leaves, unprocessed organic fruits, and unprocessed organic vegetables were used.

Ten small, equally spaced, ⅜-inch diameter holes were drilled on the bottom of the composter to serve as air passages. A tray was placed along the length of the box on the ground. This was where the composter was stationed. The composter was not in direct sunlight. Two 8 cm wood planks were evenly spaced and positioned on the tray. The composter rested on the wood.

The weight of the 5 cm×5 cm×0.2 cm bioplastic sheet was recorded. A string two times the length of the box was tied to the bioplastic. The other end of string was tied to a piece of paper with the bioplastic ID labeled on it.

The box was filled with alternating layers of brown materials (dried and carbon-rich), soil, and green materials (damp and nitrogen-rich). Near the middle of the box, in a soil section, the bioplastics were placed with the ID label outside the box. The box was filled of repeating layers until the top. The composter was placed on the 8 cm wooden planks on top of the tray.

For aeration, the composter was mixed every second day. The compost process continued for 21 days, at the end of which the bioplastics were carefully removed and let dry for two days. Excess dirt around the bioplastic was removed. The bioplastic was weighed and the weight was recorded. Using the final and initial weights of the bioplastic, the percent mass decrease was calculated using the following formula: Percent mass loss=(initial mass−final mass)/final*100.

In general, higher pumpkin and glycerin content yielded higher biodegradability. The percent mass loss of petroleum based plastic is only 10%, while, that of pumpkins based bioplastic is 18%.

What claimed is:

1. A method of making pumpkins based bioplastic, the method comprising: Separating pumpkins into flesh and seeds, and extracting organic glycerin to react with pumpkin flesh, and producing bioplastic.

2. The method of claim 1, wherein processing pumpkin seed by extracting organic glycerin by a transesterification reaction using pumpkin seed oil with methanol.

3. The method of claim 2, wherein a catalyst is added into methanol.

4. The method of claim 2, wherein a catalyst is sodium hydroxide.
